(19) United States
(12) Patent Application Publication
(10) Pub. No.: US 2015/0335009 A1
(43) Pub. Date: Nov. 26, 2015

(54) Extracorporeal Membrane Oxygenation Y-Piece Connector

(71) Applicant: Gift of Life Michigan, Ann Arbor, MI (US)

(72) Inventors: Joshua ANGEL, Sylvania, OH (US); Richard PIETROSKI, Ann Arbor, MI (US)

(21) Appl. No.: 14/718,770

(22) Filed: May 21, 2015

Related U.S. Application Data
(60) Provisional application No. 62/001,940, filed on May 22, 2014.

(51) Int. Cl. A01N 1/02; A61M 39/10

(52) U.S. Cl. CPC A01N 1/021; A01N 1/0247; A01N 1/0226; A61M 39/105

(57) ABSTRACT

A connector for an extracorporeal support (ECS) circuit including a first port, a second port, a third port, and a fourth port. The first port includes a first connector configured to couple with a cannula inserted within a femoral artery of a body. The second port includes a second connector configured to couple with an ECS tube of an ECS circuit. The third port includes a third connector configured to couple with a flush tube of an in situ flush. The fourth port is configured to receive therethrough an aortic occlusion balloon and a catheter. The connector is a single, monolithic tubular member

### CROSS-REFERENCE TO RELATED APPLICATION

This application claims the benefit of U.S. Provisional Application No. 62/001,940 filed on May 22, 2014, the entire disclosure of which is incorporated herein by reference.

### FIELD

The present disclosure relates to an extracorporeal membrane oxygenation support Y-piece connector.

### BACKGROUND

This section provides background information related to the present disclosure, which is not necessarily prior art.

Various devices and methods for preserving organs for transplant postmortem are known. For example, it is known to maintain blood flow to organs postmortem in order to perfuse the organs and make them suitable for being transplanted. While existing devices and methods for perfusing organs are suitable for their intended use, they are subject for improvement.

### DRAWINGS

The drawings described herein are for illustrative purposes only of selected embodiments and not all possible implementations, and are not intended to limit the scope of the present disclosure.

FIG. 1 is a planar view of a Y-piece connector according to the present teachings for an extracorporeal support (ECS) circuit;

FIG. 2 illustrates the Y-piece connector of FIG. 1 in an ECS circuit according to the present teachings;

FIG. 3 is a planar view of an additional Y-piece connector according to the present teachings for an ECS circuit; and

FIG. 4 illustrates an introducer of the Y-piece connector of FIG. 3 detached from the Y-piece connector.

Corresponding reference numerals indicate corresponding parts throughout the several views of the drawings.

### DETAILED DESCRIPTION

Example embodiments will now be described more fully with reference to the accompanying drawings.

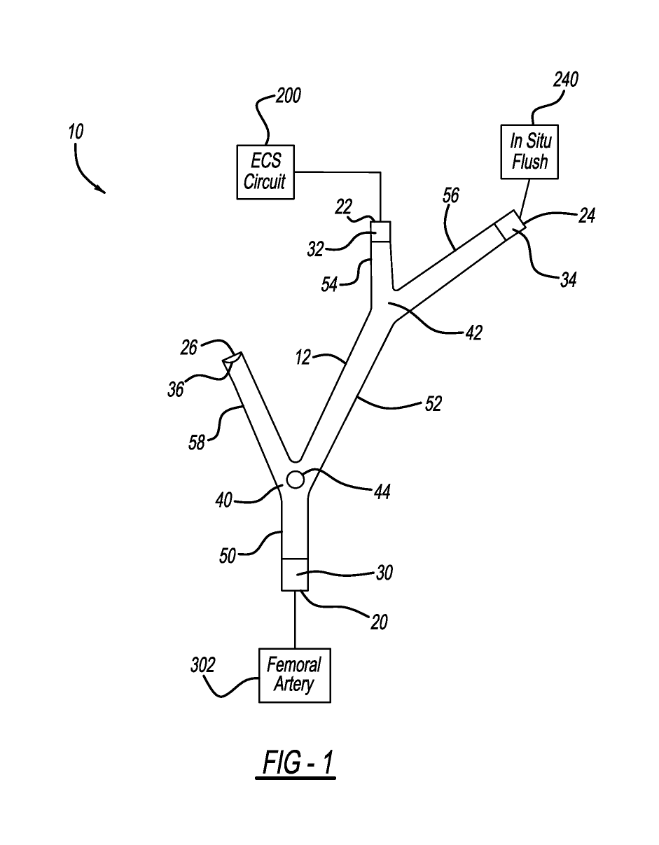

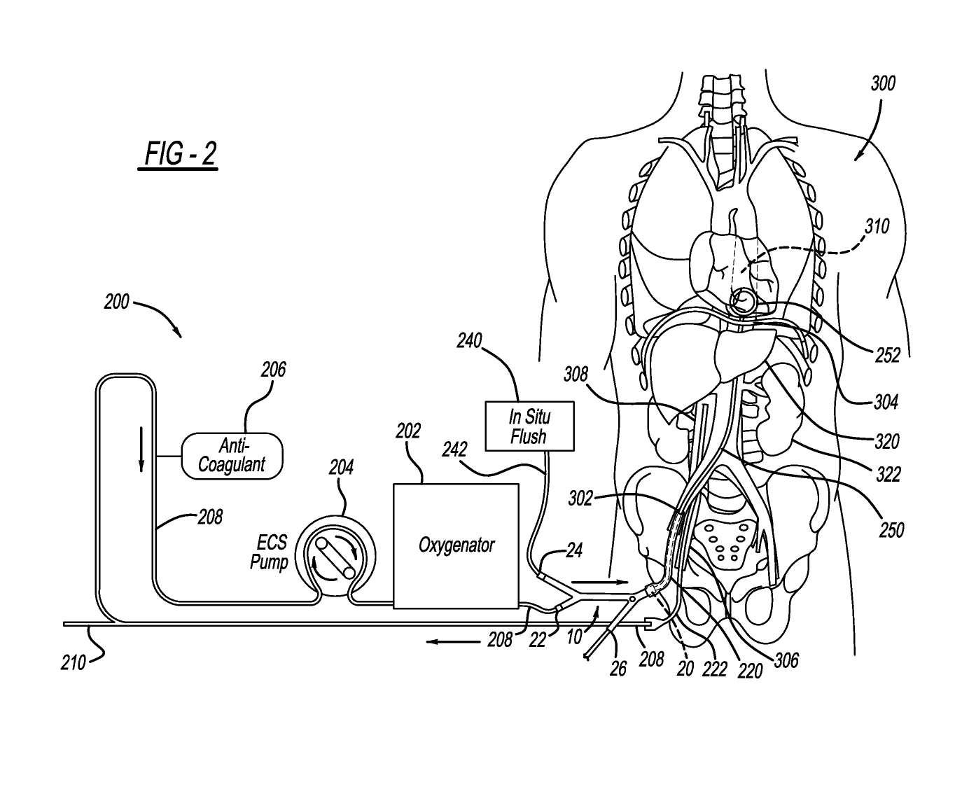

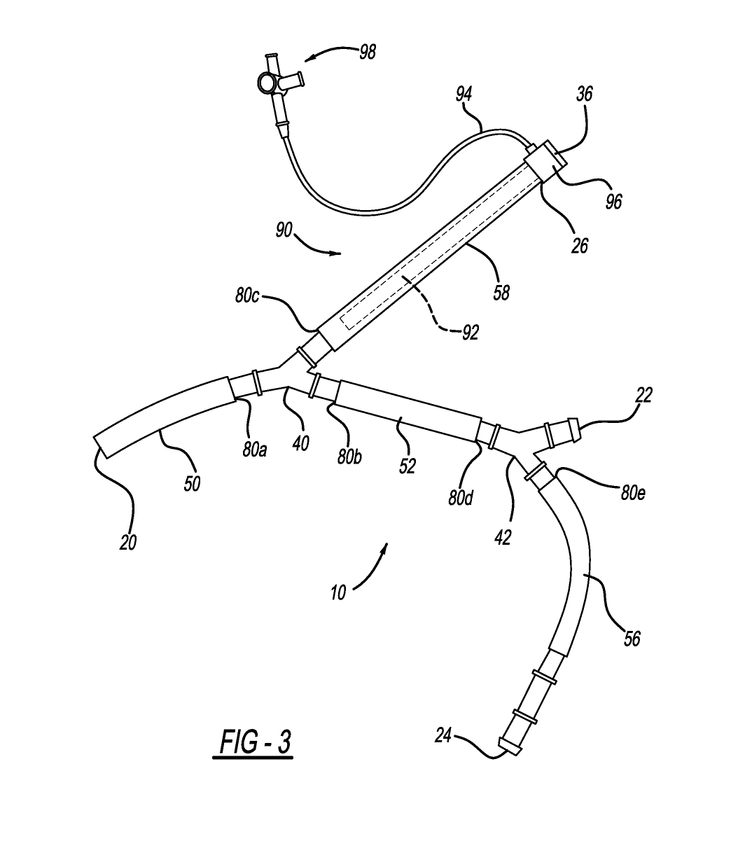

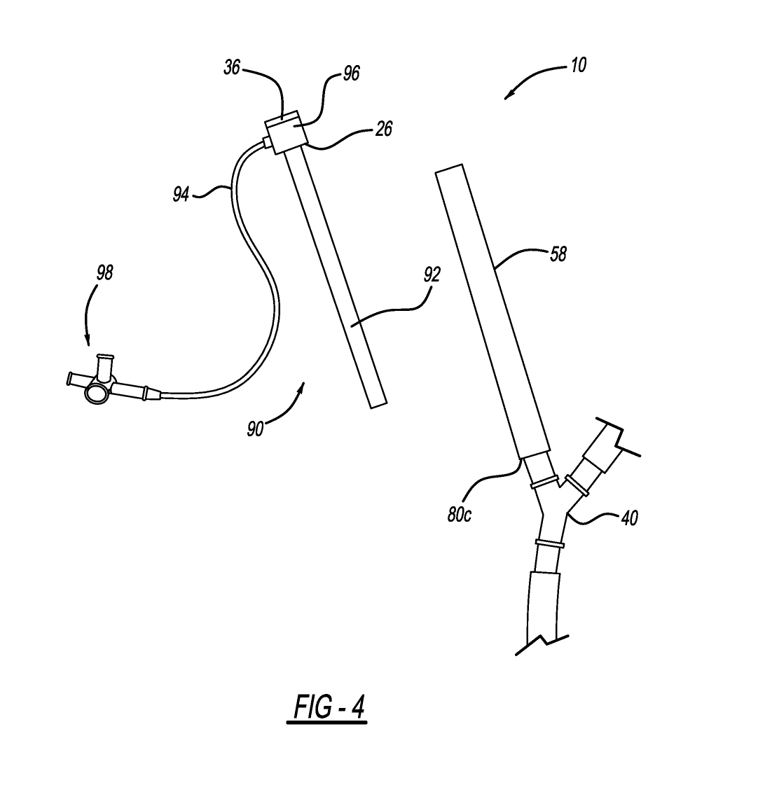

FIG. 1 illustrates a Y-piece connector 10 according to the present teachings for use with an extracorporeal support (ECS) circuit 200 (FIG. 2), such as an extra extracorporeal membrane oxygenation support circuit. Although the connector 10 is generally Y-shaped, the connector 10 can have any suitable shape or configuration. The connector 10 is generally a single tubular member 12, which is monolithic and does not include internal connectors. The connector 10 can be molded as one single piece. Alternatively, the connector 10 may not be a single piece, but may have a plurality of pieces connected together in any suitable manner, such as described herein and illustrated FIGS. 3 and 4. The connector 10 can be made of any suitable material, such as any suitable polymer.

The connector 10 generally includes a first port 20, a second port 22, a third port 24, and a fourth port 26, each of which are defined by the tubular member 12. Proximate to or at the first port 20 is a first connector 30. Proximate to or at the second port 22 is a second connector 32. Proximate to or at the third port 24 is a third connector 34. A valve 36 is at the fourth port 26. An additional connector may be included at or proximate to the fourth port 26.

The first connector 30, the second connector 32, the third connector 34, and any connector at or proximate to the fourth port 26 can each be any suitable connector configured to couple the Y-piece connector 10 to various devices, such as portions of the ECS circuit 200. Each one of the first connector 30, the second connector 32, the third connector 34, and any connector at or proximate to the fourth port 26 can be a universal connector configured to couple with any suitable tube, cannula, or any other device. For example, the first connector 30 can be configured to couple the Y-piece connector 10 to a cannula 220 inserted into a body 300 and leading to a femoral artery 302 (see FIG. 2). The second connector 32 can be configured to couple the Y-piece connector 10 to the ECS circuit 200, such as by way of an ECS tube 208. The third connector 34 can be configured to couple the Y-piece connector 10 to a flush tube 242 extending from an in situ flush 240.

The valve 36 at the fourth port 26 can be any suitable valve configured to receive therethrough a catheter 250 including an aortic occlusion balloon 252 at a distal end thereof (see FIG. 2). The valve 36 can be any suitable one-way valve configured to prevent passage of blood, for example, out from within the Y-piece connector 10.

The Y-piece connector 10 further includes a first junction 40 and a second junction 42, which can have the same dimensions, or substantially the same dimensions, as explained below. The first junction 40 includes a one-way valve 44, which can be any suitable one-way valve configured to permit release of air out from within the Y-piece connector 10, such as a suitable Luer lock. Extending between the first port 20 and the first junction 40 is a first portion 50 of the tubular member 12. The first portion 50 can have any suitable diameter, such as three-eighths of an inch. Extending between the first junction 40 and the second junction 42 is a second portion 52 of the tubular member 12. The second portion 52 can have any suitable diameter, such as three-eighths of an inch. Extending between the second junction 42 and the second port 22 is a third portion 54 of the tubular member 12. The third portion 54 can have any suitable diameter, such as three-eighths of an inch or a quarter inch. Extending between the second junction 42 and the third port 24 is a fourth portion 56 of the tubular member 12. The fourth portion 56 can have any suitable diameter, such as three-eighths of an inch or a quarter inch. Extending between the first junction 40 and the fourth port 26 is a fifth portion 58 of the tubular member 12, which is generally an introducer for receiving the catheter 250 and the aortic occlusion balloon 252 therethrough. The fifth portion 58 can have any suitable diameter such as three-eighths of an inch or a quarter inch. The fifth portion 58 and the fourth portion 56 can have the same, or substantially the same, dimensions.

With reference to FIG. 2, the ECS circuit 200 and connections to the Y-piece connector 10 will now be described in further detail. The ECS circuit 200 generally includes any suitable oxygenator 202, any suitable ECS pump 204, any suitable anticoagulant 206 (such as Heparin), and a drain tube 210. The Y-piece connector 10 is coupled to the oxygenator 202 with the ECS tube 208, which extends from the second port 22 of the Y-piece connector 10 to the oxygenator 202. From the oxygenator 202 another portion of the ECS tube 208 extends to the ECS pump 204 in order to couple the ECS pump 204 to the oxygenator 202. Another portion of the ECS tube 208 extends to the anticoagulant 206. From the anticoagulant 206, an additional portion of the ECS tube 208 extends into the body 300, and specifically into a femoral vein 306 to vena cava 308. Between the body 300 and the anticoagulant 206 is the drain tube 210 of the ECS circuit 200. From the third port 24 of the Y-piece connector extends the flush tube 242, which connects the Y-piece connector 10 to the in situ flush 240. The first port 20 of the Y-piece connector 10 is coupled to a cannula 220, which extends into the femoral artery 302.

Prior to death or just after, the cannula 220 is inserted into the femoral artery 302 and the Y-piece connector is coupled to the cannula 220. Also, a venous cannula 222 is inserted into the femoral vein 306 to the vena cava 308, and the ECS tube 208 is coupled to the venous cannula 222. Prior to death or just after, the catheter 250 including the aortic occlusion balloon 252 is inserted through the valve 36 at the fourth port 26 of the Y-piece connector 10. The catheter 250 is advanced through the fifth portion 58 and the first portion 50 of the Y-piece connector 10 such that the catheter 250 and aortic occlusion balloon 252 extend out through the first port 20, through the cannula 220, and into the femoral artery 302. The aortic occlusion balloon 252 is positioned at or slightly superior to the diaphragm 304 in aorta 310.

After death, the aortic occlusion balloon 252 is inflated, and the ECS circuit 200 is activated. Activation of the ECS pump 204 pumps the anticoagulant 206 and blood from the body 300 through the oxygenator 202, through the Y-piece connector 10, into the femoral artery 302, and to the aortic occlusion balloon 252. The aortic occlusion balloon 252 prevents blood from passing beyond the aortic occlusion balloon 252 further into the aorta 310. After contacting the aortic occlusion balloon 252, blood flows to various organs of the body 300, such as the liver 320, the kidney 322, and the spleen in order to perfuse the organs, and preserve the organs for transplantation. From the organs, blood empties into the vena cava 308. From the vena cava 308, blood is pumped back to the ECS circuit 200 by way of the venous cannula 222 to the ECS tube 208, and again back through the body 300 and the organs in the same manner described above. After perfusion of the organs is complete and the aorta 310 has been cross-clamped, the organs may be transplanted. Blood is then drained from the body 300 and the in situ flush 240 is introduced to the body 300 to flush all remaining blood from the body 300, which is drained out of the ECS circuit 200 through the drain tube 210. This procedure is often performed in an operating room.

With reference to FIGS. 3 and 4, the Y-piece connector 10 need not be a single monolithic piece, but may rather include a plurality of internal connections connecting the different portions thereof together. For example, a first connection 80a can be between the first portion 50 and the first junction 40. A second connection 80b can be between the first junction 40 and the second portion 52. Connection 80c can be between the first junction 40 and the fifth portion 58 (introducer). Connection 80d can be between the second portion 52 and the second junction 42. Connection 80e can be between the second junction 42 and the fourth portion 56. The connections 80a-80e can be any suitable connections, such as Luer lock connectors. While the Y-piece connector 10 including the connections 80a-80e is suitable for its intended use, elimination of the connectors 80a-80e, such as to provide the monolithic or single tubular member 12 of the Y-piece connector 10 as illustrated in FIG. 1, can advantageously eliminate any possibility of air undesirably entering the Y-piece connector through the connections 80a-80e.

With continued reference to FIGS. 3 and 4, the fifth portion 58 can be elongated, as compared to the fifth portion 58 of FIGS. 1 and 2. An introducer assembly 90 can also be included. The introducer assembly 90 can include an elongated tube 92, a flexible tube 94 extending from a hub 96, and a valve 98 at a distal end of the flexible tube 94. The introducer assembly 90 can be any suitable hemostatic introducer assembly, such as any suitable hemostatic introducer assembly provided by St. Jude Medical, such as part number 406136. As illustrated in FIG. 3, the elongated tube 92 is inserted into the elongated fifth portion 58, and the hub 96 is coupled to the fifth portion 58 at the fourth port 26. The hub 96 includes the one way valve 36, which is configured to receive therethrough the catheter 250 and the aortic occlusion balloon 252. The introducer assembly 90 is optional and need not be included. For example, the Y-piece connector 10 as illustrated in FIGS. 1 and 2 does not include the introducer assembly 90.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A connector for an extracorporeal support (ECS) circuit comprising:
a first port including a first connector configured to couple with a cannula inserted within a femoral artery of a body;
a second port including a second connector configured to couple with an ECS tube of an ECS circuit;
a third port including a third connector configured to couple with a flush tube of an in situ flush; and
a fourth port configured to receive therethrough an aortic occlusion balloon and a catheter;
wherein the connector is a single, monolithic tubular member.

2. The connector of claim 1, wherein each one of the first connector, the second connector, and the third connector is a universal connector configured to couple with a variety of tubular members of different types.

3. The connector of claim 1, wherein the connector includes a one-way valve configured to permit release of air from within the connector.

4. The connector of claim 1, wherein the connector includes a first junction and a second junction.

5. The connector of claim 4, further comprising a first portion of the connector extending from the first port to the first junction, a second portion of the connector extending from the first junction to the second junction, a third portion of the connector extending from the second junction to the second port, a fourth portion of the connector extending from the second junction to the third port, and a fifth portion of the connector extending from the first junction to the fourth port.

6. The connector of claim 5, wherein the first junction is between the first portion, the second portion, and the fifth portion of the connector.

7. The connector of claim 6, wherein the second junction is between the second portion, the third portion, and the fourth portion of the connector.

8. The connector of claim 1, wherein the fourth port includes a one-way valve configured to receive the aortic occlusion balloon and the catheter therethrough.

9. The connector of claim 5, wherein the first portion, the second portion, and the fifth portion have a diameter of three-eighths of an inch.

10. A method for perfusing organs of a body upon death comprising:
coupling a first port of a connector for an extracorporeal support (ECS) circuit to a cannula extending to a femoral artery of the body;
coupling a second port of the connector to an oxygenator of the ECS circuit;
coupling a third port of the connector to a flush tube of an in situ flush;
coupling an ECS tube of the ECS circuit to a venous cannula that has been inserted into a femoral vein of the body, the ECS tube in fluid communication with the oxygenator and an ECS pump of the ECS circuit;
inserting a catheter with an aortic occlusion balloon at an end thereof through the fourth port such that the aortic occlusion balloon passes out of the first port, through the cannula, and into the femoral artery;
positioning the aortic occlusion balloon in an aorta superior to a diaphragm of the body;
inflating the aortic occlusion balloon after death; and
activating the ECS pump after death to pump blood into the connector through the second port, out of the connector through the first port, and into the femoral artery to the aortic occlusion balloon positioned to direct blood to organs of the body including a liver, kidney, and spleen to perfuse the organs.

11. The method of claim 10, further comprising introducing an anticoagulant into the blood pumped with the ECS pump.

12. The method of claim 11, wherein the anticoagulant is heparin.

13. The method of claim 10, further comprising clamping the aorta after death and introducing a flush into the body from the in situ flush.

14. The method of claim 10, further comprising releasing air from within the connector through a one-way valve of the connector.

15. The method of claim 10, further comprising releasing air from within the connector through a one-way valve of the connector arranged between the first port and the fourth port.
